(19) United States
(12) Patent Application Publication
(10) Pub. No.: US 2026/0174590 A1
(43) Pub. Date: Jun. 25, 2026

(54) EXTERNAL FEMALE URINARY INCONTINENCE MANAGEMENT DEVICES

(71) Applicant: Thara Satish, Riverview, FL (US)

(72) Inventor: Thara Satish, Riverview, FL (US)

(21) Appl. No.: 19/427,482

(22) Filed: Dec. 19, 2025

Related U.S. Application Data
(60) Provisional application No. 63/735,906, filed on Dec. 19, 2024.

(51) Int. Cl. A61F 5/455; A61F 5/44; A61M 1/00

(52) U.S. Cl. CPC A61F 5/455; A61F 5/4401; A61M 1/60; A61M 2202/0496

(57) ABSTRACT

A female external urinary incontinence management device has a concave panel with a perpendicularly extending rim along its periphery and an outwardly extending flange along the top of the rim. A drainage outlet in the panel may be connected to a drainage to from a urine suction. An absorbent pad having a thickness less the height of the rim lies flush against the panel over the drainage outlet. When applied to a patient such that the flange lies flush against the patient's pubic region, the device forms a void such that the pad does not rub against the vulva.

### CROSS-REFERENCE TO RELATED APPLICATIONS

This application claims priority to U.S. Provisional Application Ser. No. 63/735,906 filed on Dec. 19, 2024, the contents of which are hereby incorporated in their entirety.

### STATEMENT REGARDING FEDERALLY SPONSORED RESEARCH OR DEVELOPMENT

Not Applicable.

### NAMES OF PARTIES TO A JOINT RESEARCH AGREEMENT

Not Applicable

### REFERENCE TO SEQUENCE LISTING, A TABLE, OR A COMPUTER PROGRAM LISTING APPENDIX SUBMITTED ON A COMPACT DISC AND INCORPORATION-BY-REFERENCE OF THE MATERIAL

Not Applicable.

### COPYRIGHT NOTICE

A portion of the disclosure of this patent document contains material which is subject to copyright protection. The copyright owner has no objection to the facsimile reproduction by anyone of the patent document or the patent disclosure after formal publication by the U.S. Patent Office, as it appears in the U.S. Patent and Trademark Office patent file or records, but otherwise reserves all copyright rights whatsoever.

### BACKGROUND OF THE INVENTION

### Field of the Invention

The present invention relates to a female external catheter. More particularly, the invention relates to an external female urinary incontinence management device for removing urine without irritating skin.

### Description of the Related Art

Incontinence in bed-bound and/or wheelchair-bound patients can lead to bedsores, skin excoriation, and non-healing wounds in and around the sacrum and perineal areas. One common method for preventing this is to insert a catheter in the patient. However, utilizing a catheter requires medical professionals, and can lead to several complications such as infection. They also must be replaced frequently. External female catheters often provide a safer, more comfortable, and less invasive urine management option for many patients, particularly when the goal is to manage incontinence while minimizing infection and injury risks.

External female catheters, also referred to as female external urinary collection devices, offer several practical and clinical benefits, especially compared with indwelling, internal catheters. They reduced risk of urinary tract infections (UTIs), significantly lower the risk of catheter-associated UTIs compared with Foley catheters, improve patient comfort, avoid introducing bacteria into the urethra and bladder, cause less pain, irritation, and anxiety, and are often better tolerated for long-term or overnight use. They also reduce the risk of urethral and bladder injury and eliminate trauma such as urethral erosion, strictures, or bladder spasms associated with internal catheters. When properly managed, they also improve skin integrity.

Many external catheter systems use gentle suction to draw urine away, helping keep skin dry and reducing exposure to moisture, thus lowering the risk of incontinence-associated dermatitis and pressure injuries. They also provide better mobility and dignity, are less restrictive than indwelling catheters, and usually allow some patients to sit up, reposition, or move more freely. They are perceived as less invasive and more dignified.

However, external incontinence devices also require care when used. Their effectiveness can depend on proper positioning and patient anatomy. Skin monitoring is still important to prevent irritation or pressure injury. Most female external catheters are designed to include a porous absorbent material that presses against a patient's pubic area, and some are designed to be at least partially inserted between the labia. This is often considered necessary to properly wick away moisture, which is removed from the device using suction. This constant contact between the wicking material and the patients skin can itself cause skin irritation and sores. This is particularly true in regard to the sensitive skin under the labia. This can lead to urine coming into contact with sores on the skin, thus causing discomfort and infection. Furthermore, many external catheters are not designed to be easily adjusted to fit a particular patient. As a result, they can be inadvertently moved from their proper position, causes leakage, infection and embarrassment.

The above-described deficiencies of today's systems are merely intended to provide an overview of some of the problems of conventional systems, and are not intended to be exhaustive. Other problems with the state of the art and corresponding benefits of some of the various non-limiting embodiments may become further apparent upon review of the following detailed description.

In view of the foregoing, it is desirable to provide an external female incontinence management device that reliable rests in a desired position on the patient and does not require constant contact between the patient's skin and an absorbent material.

### BRIEF SUMMARY OF THE INVENTION

Disclosed is a female external urinary incontinence management device that is relatively inexpensive, easy to use, cost-effective and provides comfort and dignity for the patients. A female external urinary incontinence management device has a concave panel with a perpendicularly extending rim along its periphery and an outwardly extending flange along the top of the rim. A drainage outlet in the panel may be connected to a drainage to from a urine suction. An absorbent pad having a thickness less the height of the rim lies flush against the panel over the drainage outlet. When applied to a patient such that the flange lies flush against the patient's pubic region, the device forms a void such that the pad does not rub against the vulva.

In one embodiment, the external female urinary incontinence management device has a panel with a concave interior side defined by a rim extending substantially perpendicularly from a periphery of the panel. A flange extends outward from a top of the rim substantially parallel to the panel. A drainage outlet in the concave base of the tray is configured to removably connect to a drainage tube in fluid communication with a vacuum pump. A removable, absorbent pad lies flush against the concave base, and the thickness of the absorbent pad is less than the height of the rim.

It is therefore an object of the present invention to provide an incontinence management device that that improves the quality of life of both the patient and the caregiver, and leads to fewer diaper changes and faster wound healing.

These and other objects and advantages of the present invention will become apparent from a reading of the attached specification and appended claims. There has thus been outlined, rather broadly, the more important features of the invention in order that the detailed description thereof that follows may be better understood, and in order that the present contribution to the art may be better appreciated. There are features of the invention that will be described hereinafter and which will form the subject matter of the claims appended hereto.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an environmental view of a female external urinary incontinence management device in accordance with the principles of the invention;

FIG. 2 is another environmental view of a female external urinary incontinence management device in accordance with the principles of the invention;

FIG. 3 is a perspective view of a female external urinary incontinence management device in accordance with the principles of the invention;

FIG. 4 is a front elevation view of a female external urinary incontinence management device in accordance with principles of the invention;

FIG. 5 is a front elevation view of an absorbent pad for a female external urinary incontinence management device in accordance principles of the invention;

FIG. 6 is a side elevation view of an absorbent pad and a female external urinary incontinence management device in accordance with principles of the invention;

FIG. 7 is a front elevation view of an alternative embodiment of a female external urinary incontinence management device in accordance with principles of the invention;

FIG. 8 is a cross-sectional view of an alternative embodiment of a female external urinary incontinence management device accordance with principles of the invention;

FIG. 9 is a cross-sectional view of another alternative embodiment of a female external urinary incontinence management device in accordance with principles of the invention;

FIG. 10 is a cross-sectional view of another alternative embodiment of a female external urinary incontinence management device in accordance with principles of the invention;

FIG. 11 is a front elevation view of an alternative embodiment of a female external urinary incontinence management device in accordance with principles of the invention;

FIG. 12 is an environmental view of an alternative embodiment of a female external urinary incontinence management device being correctly positioned in accordance with principles of the invention;

FIG. 13 is an environmental view of a correctly positioned alternative embodiment of a female external urinary incontinence management device in accordance with principles of the invention.

### DETAILED DESCRIPTION

The invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The disclosed subject matter is described with reference to the drawings, wherein like reference numerals are used to refer to like elements throughout. In the following description, for purposes of explanation, numerous specific details are set forth in order to provide a thorough understanding of the various embodiments of the subject disclosure. It may be evident, however, that the disclosed subject matter may be practiced without these specific details. In other instances, well-known structures and devices may be shown in block diagram form in order to facilitate describing the various embodiments herein.

Various embodiments of the disclosure could also include permutations of the various elements as if each dependent claim was a multiple dependent claim incorporating the limitations of each of the preceding dependent claims as well as the independent claims. Unless explicitly stated otherwise, such permutations are expressly within the scope of this disclosure. Similarly, the disclosure should be interpreted as including permutations of the various elements disclosed in the Figures, unless the various elements are clearly mutually exclusive.

Unless otherwise indicated, all numbers expressing quantities of ingredients, dimensions reaction conditions and so forth used in the specification and claims are to be understood as being modified in all instances by the term “about”. The term “a” or “an” as used herein means “at least one” unless specified otherwise. In this specification and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

For ease of understanding, the following definitions will apply throughout this description; however, no definition should be regarded as being superceding any art-accepted understanding of the listed terms.

Disclosed is a female external urinary incontinence management device designed to prevent irritation to the patient's skin by creating a void between the absorbent pad in the patient's skin. This prevents the absorbent pad from sticking to the skin, abrading it, or otherwise causing discomfort while also keeping the patient dry and free from urine. The device has a concave panel substantially matching the convex to the of a typical patients pubic area. The panel has a rim around its periphery that extends perpendicularly from the panel. A flange extends outward from the top of the rim. The entire devices preferably unitary made for example from a silicone mold. The absorbent pad is positioned flush against the panel while the flange is flush against the patient. The height of the rim is greater than the thickness of the absorbent pad, thus creating a small void. A drainage outlet in the panel allows a drainage tube connected to a suction pump to be affixed to the incontinence management device to remove urine when necessary. The flange on opposite sides of the device may be placed within the crease between a patient's thighs and abdomen to help secured in place.

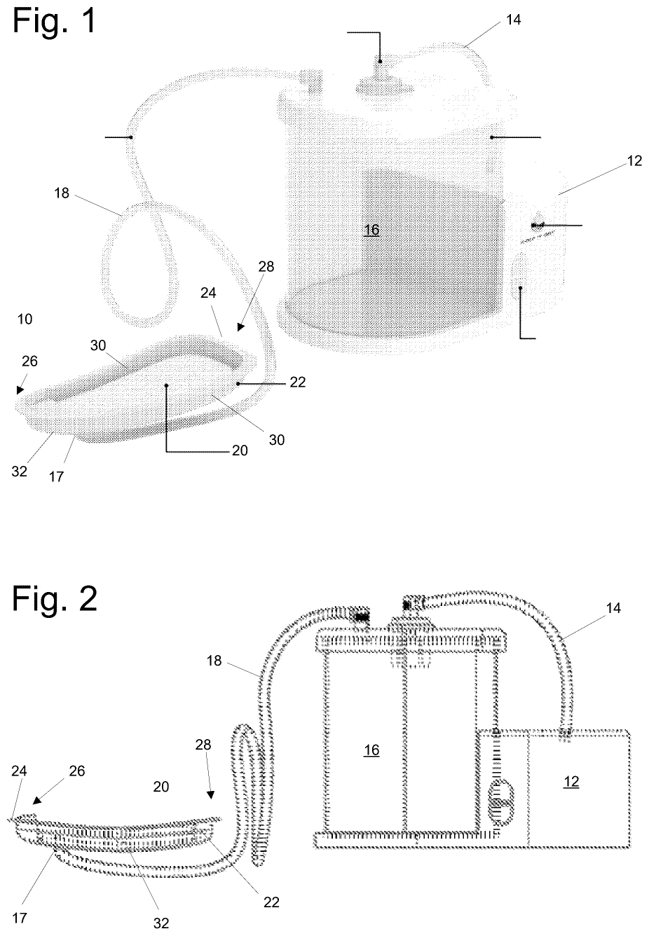

FIGS. 1 and 2 show an external female urinary incontinence device 10 connected to a typical urine suction device 12. The urine suction device 12 includes an internal suction pump, not shown, connected to and providing suction through a suction tube 14 in communication with the urine storage chamber 16. A drainage tube 18 extends from the urine storage chamber 16 to a drainage outlet 17 on the external side of the incontinence device 10. The incontinence device 10 has a concave panel 32 and contains an absorbent removable pad 20 secured flush against the panel inside the space defined by the panel 32 and rim 22. A rim 22 extends perpendicularly from the periphery of the panel and has an outward extending flange 24. A first end 26 is narrower than the second end 28 and the sidewalls 30 of the rim taper inward as they extend from the second end 28 to the first end 26. In this embodiment, two flexible wings 25 extend partially over the absorbent pad 20 from the two corners defining the first end 26. It is generally desirable for the device 10 to be unitary and not formed by two or more parts fitted or joined together. For example, device 10 is formed from silicone in a single mold. The use of silicone allows it to be flexible but strong enough to retain its shape, and soft enough to be comfortable for the patient.

In use, the incontinence device 10 is placed over the pubic region of a female, inside a diaper or underwear, such that it substantially covers the vulva. The first end 26 is placed at the bottom end of the vulva and the second end 28 is positioned to fully cover the labia. When the suction pump is activated, the suction provided through the drainage tube 18 further pulls the absorbent pad 20 against the panel 32, causes the flange 24 to form a seal with the patient's skin surrounding the vulva, and removes any moisture, particularly urine, from the patient's pubic area. The rim 22 has a height that is equal to or greater than the thickness of the absorbent pad 20. As a result, only the flange 24 is in contact with a patient's skin, and it is only firmly held against the skin when the suction devices activated. This essentially creates a seal along the flange. Furthermore, nothing is permanently pressed against the vulva or inside the labia.

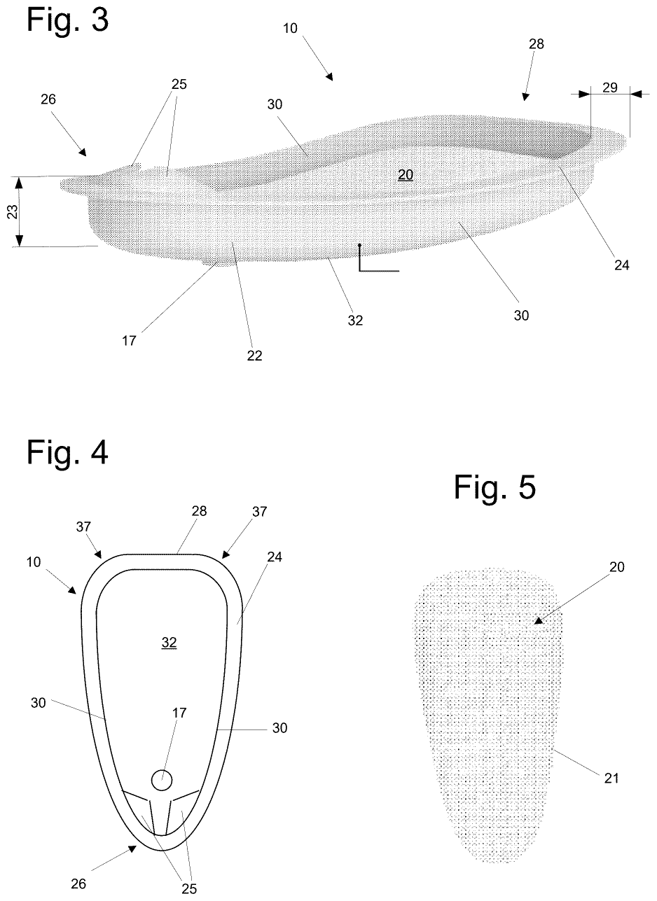

FIG. 3 shows the external female urinary incontinence management device 10 in more detail. The panel 32, with the rim 22, forms a concave cavity or chamber that is partially occupied by the pad 20. The concavity of the panel 32 approximates a roughly average curvature of the human body in the pubic region. The panel 32, rim 22 and flange 24 are preferably formed as a single unitary components. The drainage outlet 17 is removably attachable to a drainage tube as shown in FIGS. 1 and 2. The rim 22 has a substantially constant height 23 and the outwardly extending flange 24 similarly has a substantially constant width 29 that is approximately equal to or less than the height 23 of the rim 22. The two wings 25 may assist in positioning the first end 26 such that the lower end of the vulva is within a region defined by the rim 22. Wings 25 also assist in securing the pad 20 inside the device flush against the interior side of the panel 32.

FIG. 4 shows the device 10 with the pad 20 removed, while FIG. 5 shows the removed absorbent pad 20. The removable absorbent pad 20 is formed from a porous material and may include a semirigid internal frame such that it retains its shape and its peripheral edge 21 may form a friction fit with the inner walls of the rim 22, including the sidewall regions of the rim 30. In this embodiment, the second end 28, which is preferably positioned above the top of the vulva, is substantially straight, while the sidewalls of the rim 30 form a curved tapers inward to meet at the first end 26. The first in 26 and the rim sidewalls 30 thus form a curved first region of the rim while the second end 28 forms a straight second region of the rim. This first region in this embodiment has the approximate shape of a parabolic curve and is joined to the second region at two curved corners 37. The drainage outlet 17 is positioned at a point mathematically defined as the focus of the parabolic curve formed by the first region of the rim.

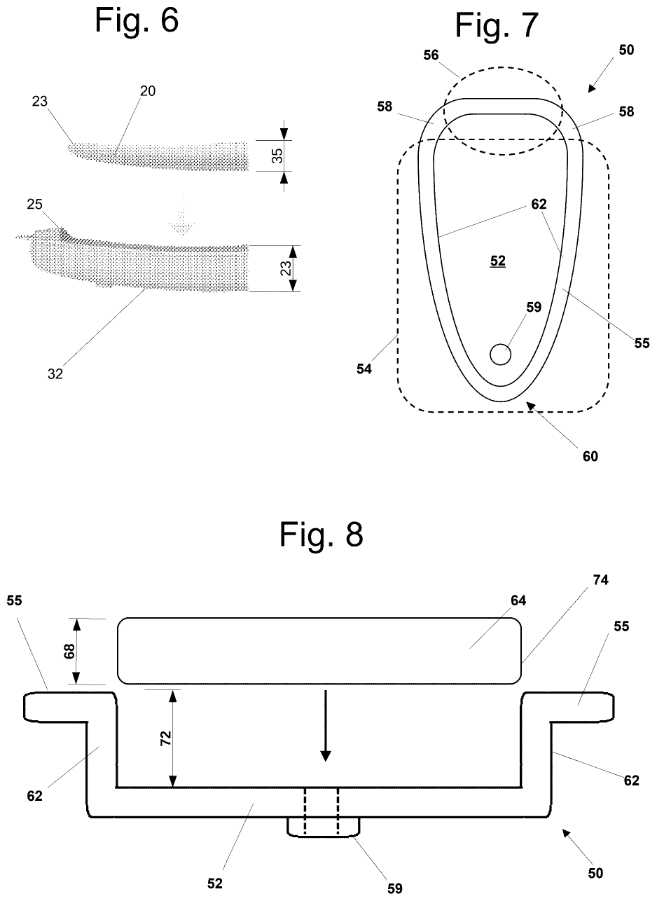

FIG. 6 shows the thickness 35 of the pad 20 may be less than the height 23 of the rim 22. The pad 20 has a first end 23 that curves slightly upward to both match the concavity of the panel 32 and also to form a secure friction fit underneath the wings 25. The upward curving of the first in 23 of the pad 20 also ensures that the pad 20 remains flush against the panel 32 and is not pressed against a patient's skin or labia, although it may be in gentle contact with the skin or labia.

FIG. 7 shows an alternative embodiment of an external female urinary incontinence management device 50 without an absorbent pad inserted into it. Device 50, like device 10, has a concave panel 52 surrounded by a rim having a flange 55. The rim includes a first region 54 and a second region 56, which connect to one another at rounded corners 58. The first region 54 is curved and the drainage outlet 59 is positioned at the center of curvature of the lower end 60, where the two rim sidewalls 62 join, of the first region 54. The rim sidewalls 62 curve and taper inward as they extend down to the lower end 60. Those skilled in the art will appreciate that the sidewall 62 may optionally be straight to form more of a V-shaped rather than a U-shaped of devices 10 and 50. It is generally desirable to have rounded corners for the patient's comfort, but sharper corners will still allow the device to function adequately.

FIG. 8 shows the female external urinary incontinence management device 50 with an absorbent pad 64 being lowered into it. The absorbent pad has a thickness 68 that is equal to or less than the height 72 of the rim 62. The flange 55 has a width comparable to the height 72 of the rim 62. The absorbent pad 64 has a width slightly larger than the width of the panel 52 such that its periphery 74 may form a friction fit with the rims 62 and secure it in place inside the device 50.

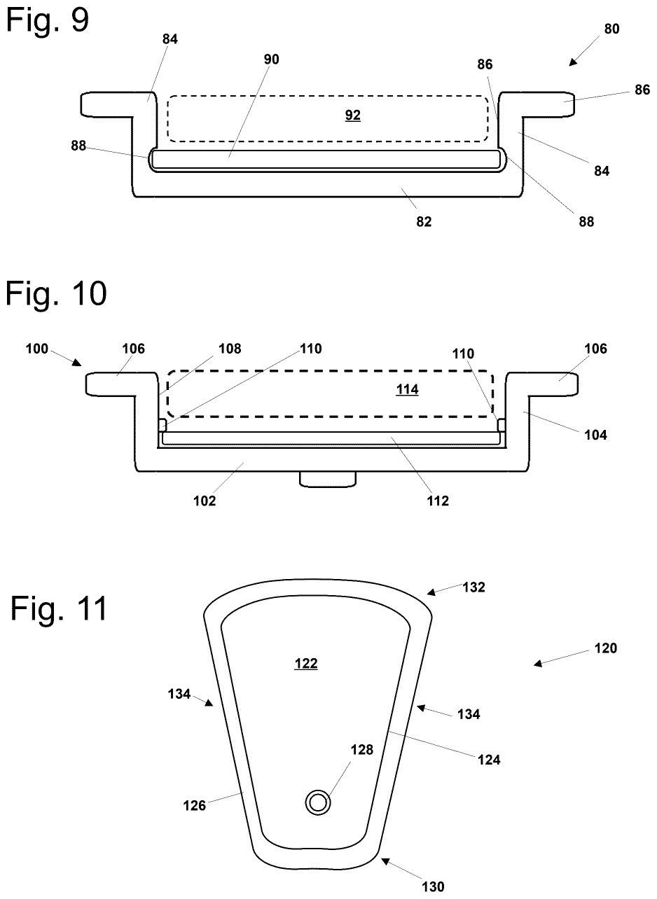

FIGS. 9 and 10 show additional an alternative mechanisms for securing and absorbent pad flush against the panel of a device to create space between them and the flanges of the rim. FIG. 9 shows a female external urinary incontinence management device 80 having a panel 82 with a perpendicularly extending rim 84 along its entire periphery, and an outwardly extending flange 86 at the top of the rim 84. In this embodiment, the inside wall 86 of the rim 84 has a channel 88 running along the rim 84 adjacent to the panel 82. The absorbent pad 90 has a width sized to fit within the channel, thereby holding it in place against the panel 82. Because the pad 90 has a thickness less than the height of the rim 84, the device 80 forms a void 92 between the pad 90 and the flanges 86. This void 92 is an air pocket between the patient and the absorbent pad. When the device is connected to a suction pump, the patient's skin between the flanges 86 remains dry and untouched by the absorbent pad 90. Similarly, FIG. 10 shows a device 100 having a panel 102 with a perpendicularly extending rim 104 along its periphery and a flange 106 extending outward from the rim 104. In this embodiment, the inner wall 108 of the rim includes a plurality of tabs 110 extending inward. When a pad 112 is positioned flush against the panel 102, the tabs 110 secure it in place. When the flange 106 is flush against a patient's skin, the device forms a thin void 114 between the absorbent pad 112 and the patient. It may be desirable to utilize a pad as shown in FIGS. 9 and 10 that has a thickness less than the height of the rim for patients with particularly sensitive skin. In all of these embodiments, the flanges are configured to lie flush against the patient's skin, surrounding the vulva. This prevents urine or other fluids from leaking out of the device while also ensuring that the device effectively captures and removes fluid with rubbing against or abrading sensitive regions of the patient.

FIG. 11 shows an alternative embodiment of a female external urinary incontinence management device 120. Like the devices of the other embodiments shown herein, device 120 includes a concave panel 122 a rim 124 extending perpendicularly along its periphery, and an outward extending flange 126 at the top of the rim 124. Device 120 also includes a drainage outlet 128 for removable attachment to a suction device such as the one shown in FIGS. 1 and 2. Device 120 differs primarily in its geometric shape. Device 120 has a relatively straight first end 130 and a curved second end 132. The device 120 has two substantially straight sides 134 that taper inward as they extend from the second end 132 to the first end 130. Thus device 120 has a somewhat trapezoidal shape.

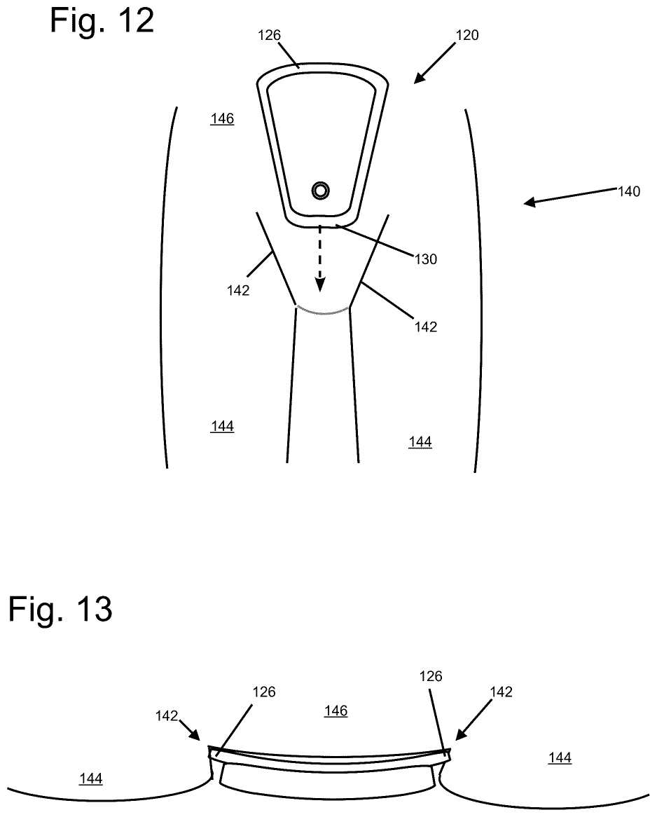

Those skilled in the art will appreciate that patients come in many different shapes and sizes. The incontinence management devices of the present invention may be readily adjusted for use by patients of varying size. FIG. 12 shows the incontinence device 120 and a method for positioning it on a patient 140. Those skilled in the art will appreciate that a crease 142 is formed between each of the thighs 144 and the abdomen 146 of a patient in the pubic region. The device 120 is placed on the lower abdomen of the patient such that the flanges lie flush against the patient's skin, and is slowly moved down into the pubic region such that the flanges 126 along the sides 134 slide into the opposing creases 142. The device is moved downward until the first end 130 is below the bottom of the vulva, as shown in FIG. 13. Because the flanges along the sides 134 form a snug fit within these creases, the device remains secured in a proper position. Often patients are also wearing underwear diapers, which further secure the device 120 in the proper position. Those skilled in the art will appreciate that the same procedure may be used with other devices shown in contemplated herein.

Whereas, the present invention has been described in relation to the drawings attached hereto, other and further modifications, apart from those shown or suggested herein, may be made within the spirit and scope of this invention. Those skilled in the art will appreciate that the conception, upon which this disclosure is based, may readily be utilized as a basis for the designing of other structures, methods and systems for carrying out the several purposes of the present invention. That is, the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. The descriptions of the embodiments shown in the drawings should not be construed as limiting or defining the ordinary and plain meanings of the terms of the claims unless such is explicitly indicated. The claims should be regarded as including such equivalent constructions insofar as they do not depart from the spirit and scope of the present invention.

1. An external female urinary incontinence management device comprising:
a panel having a concave interior side defined by a rim extending substantially perpendicularly from a periphery of the panel;
a flange extending outward from a top of the rim substantially parallel to the panel;
an drainage outlet in the concave base of the tray and configured to removably connect to a drainage tube in fluid communication with a vacuum pump;
a removable, absorbent pad lying flush against the concave base;
wherein the absorbent pad has a thickness that is equal to or less than a height of the rim.

2. The external female urinary incontinence management device of claim 1 wherein the rim is comprised of a first section having an elongated curve shape and a second straight section, and wherein the first section and second section intersect at rounded corners.

3. The external female urinary incontinence management device of claim 2 wherein the first section of the rim has a parabolic shape and the outlet in the panel is positioned at a focus the first section.

4. The external female urinary incontinence management device of claim 2 wherein the outlet in the panel is positioned at a center of curvature of the first section.

5. The external female urinary incontinence management device of claim 4 further comprising a channel extending at least partially along an interior side of the rim and the absorbent pad is configured to extend into the channel, thereby securing the absorbent pad flush against the panel.

6. The external female urinary incontinence management device of claim 4 further comprising a plurality of tabs extending inward from the rim and the absorbent pad is configured to extend underneath the tabs, thereby securing the absorbent pad flush against the panel.

7. The external female urinary incontinence management device of claim 4 wherein the panel has a trapezoidal shape.

8. The external female urinary incontinence management device of claim 5 wherein the height of the rim is at least twice the thickness of the absorbent pad.
